(19) United States
(12) Patent Application Publication
(10) Pub. No.: US 2010/0081912 A1
(43) Pub. Date: Apr. 1, 2010

(54) Ultrasound-Optical Doppler Hemometer and Technique for Using the Same

(75) Inventors: Edward M. McKenna, Boulder, CO (US); Clark R. Baker, JR., Newman, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/242,401

(22) Filed: Sep. 30, 2008

(51) Int. Cl. A61B 5/00; A61B 8/06

(52) U.S. Cl. 600/368; 600/454

(57) ABSTRACT

According to embodiments, a sensor assembly and/or systems for ultrasound-optical measurements may provide information related to hemodynamic parameters. An ultrasound beam may be used to generate a Doppler field for optical elements of a sensor assembly. By combining information received from ultrasound and optical elements of the sensor assembly, more accurate values for hemodynamic parameters may be determined.

### BACKGROUND

The present disclosure relates generally to medical devices and, more particularly, to sensors and systems for measuring physiological parameters of a patient.

In the field of medicine, doctors often desire to monitor certain physiological characteristics of their patients. Accordingly, a wide variety of devices have been developed for monitoring many such characteristics of a patient Such devices provide doctors and other healthcare personnel with the information they need to provide the best possible healthcare for their patients. As a result, such monitoring devices have become an indispensable part of modern medicine.

A physiological characteristic that may provide information about the clinical condition of a patient is the total concentration of hemoglobin in blood (Hb_{T}) or the hematocrit (Hct), which relates to the fraction or percentage of red cells in whole blood. The hematocrit is the fraction of the total blood volume occupied by the red blood cells, and hemoglobin is the principal active constituent of red blood cells. Approximately 34% of the red cell volume is occupied by hemoglobin.

Typically, hematocrit measurements may be performed by relatively invasive techniques that involve drawing a patient's blood and directly measuring the solid (packed-cell) fraction that remains after centrifugation of the blood. Such techniques may involve relatively labor-intensive steps that are performed by skilled healthcare providers. Other techniques may involve noninvasive estimation of the hematocrit through the optical characteristics or electrical characteristics of the tissue that is measured. While these techniques provide the advantage of not involving a drawn blood sample, the measurements rely upon algorithms that make general assumptions that may not account for individual patient anatomies.

### BRIEF DESCRIPTION OF THE DRAWINGS

Advantages of the disclosure may become apparent upon reading the following detailed description and upon reference to the drawings in which:

FIG. 1 illustrates a block diagram of a monitoring system in accordance with an exemplary embodiment;

FIG. 2 illustrates a view of an exemplary sensor assembly for probing hemodynamic parameters; and

FIG. 3 illustrates a flow chart for determining physiological parameters based on signals received from an ultrasound transducer and a photodetector.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

According to various embodiments, sensors, or sensor assemblies, and monitoring systems are provided herein that may employ optical-acoustic measurements to more accurately determine physiological parameters such as hematocrit. The sensor assemblies may be applied to a patient for determination of the physiological parameters. Sensor assemblies may include light emitters for emitting photons of light into a patient's tissue. A photodetector may be spaced apart from the emitter so that light that has penetrated to depths associated with blood vessels under the skin surface may be detected. Sensor assemblies may also include an ultrasound transducer that may be focused on a particular area of the patient's tissue to interact with the emitted light in the tissue. The emitted light that passes through the area of the ultrasound beam may undergo a Doppler shift of a detectable frequency. When the ultrasound beam is focused on an area of interest in a blood vessel, the photons that undergo the Doppler shift are, therefore, more likely to be distributed in the blood vessel and are more likely to be related to hemodynamic parameters, such as hematocrit or blood pressure. Accordingly, the signal generated at the photodetector may be processed to separate out the data more likely to be associated with hemodynamic parameters (i.e., a Doppler-shifted signal) from the data more likely to be associated with tissue absorption (i.e., signal from light that has not undergone a Doppler shift and that has been absorbed by the skin or other structures in the tissue).

From the effect of the ultrasound signal on the emitted light, determination of hemodynamic parameters may be made. For example, the Doppler shift frequency may be related to the velocity of the red blood cells in an arterial vessel. The strength of light scattered back to the detector may be related to the number of red blood cells in the artery. In addition, the ultrasonic waves used to generate the Doppler shift may also be used to generate information about the size of the vessel being probed. When the ultrasound beam is focused into a vessel, not only may the beam be used to influence the optical signal at the detector, but the beam may also be used in and of itself to provide additional information to the system related to the nature or physical characteristics of the blood vessel. For example, the ultrasound beam that is reflected back to the transducer may also generate a signal that may be processed to determine arterial size. By combining information about the size of the vessel with information generated by the detector about the velocity and concentration of the red blood cells, a more accurate determination of hemodynamic parameters may be established.

In embodiments, the addition of information about vessel size to such determinations may be advantageous in calculating parameters that have volume components. For example, hematocrit may be defined as the portion of the total volume of blood occupied by red blood cells and may be expressed as a decimal (liter/liter) value or a percentage (liter/liter×100%) value. Typically, in calculations of hematocrit, an estimated value for the vessel size, which may be determined by an average of vessel size in a large patient pool, is used in the calculation. In embodiments, rather than using an empirically derived estimated mean value for the vessel size, an ultrasonically measured value for the probed volume of interest may be substituted to provide increased accuracy for hematocrit determinations. Similarly, determination of other hemodynamic parameters that involve volume components may also benefit from using a directly measured vessel size rather than an estimated one. Such parameters may include blood pressure values and/or measures of vascular resistance. By providing measurements of various hemodynamic parameters with increased accuracy, physicians may be able to provide better patient care.

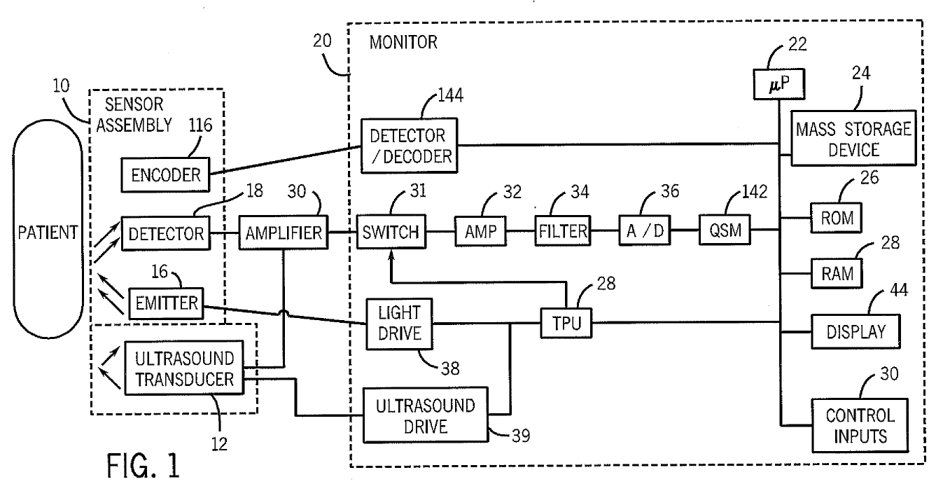

FIG. 1 illustrates a block diagram implementing a monitoring system in accordance with an exemplary embodiment. The system includes a sensor assembly to. The sensor assembly 10 is capable of providing an optical signal and an ultrasound signal to a monitor 20. The monitor 20 has a microprocessor 22 that is, in turn, capable of using the optical signal and the ultrasound signal in calculating various hemodynamic parameters, such as hematocrit, related to the signal.

The microprocessor 22 is coupled to other component parts of the monitor 20, such as a mass storage device 24, a ROM 26, a RAM 28, and control inputs 30. The mass storage device 24, the ROM 26, and/or the RAM 28 may hold the algorithms or routines used to determine the hemodynamic parameters and may store the data collected by the sensor assembly 10 for use in the algorithms. The mass storage device 24 may be any suitable device such as a solid state storage device, an optical medium (such as an optical disk) or a magnetic medium (such as a hard disk). The monitor 20 may include a display 44 for providing information to healthcare providers related to the measurements generated by the microprocessor 22.

Detected optical signals and ultrasound signals are passed from the sensor assembly 10 through one or more amplifiers 30 to the monitor 20 for processing. In the monitor 20, the signals may be amplified and filtered by amplifier 32 and filter 34, respectively, before being converted to digital signals by an analog-to-digital converter 36. The digitized signals may then be used to determine the fluid parameters and/or may be stored in RAM 28 and mass storage device 24.

A light drive unit 38 in the monitor 20 controls the timing of the optical components, such as emitters 16, in the sensor assembly 10. An ultrasound drive unit 39 may control the timing of ultrasound components, such as an ultrasonic transducer 12, in the sensor assembly 10. A time processing unit (TPU) 28 may provide timing control signals. TPU 28 may also control the gating-in of signals from detector 18 through an amplifier 30 and a switching circuit 31. Because the light that generates the optical signal may undergo a detectable Doppler shift as a result of encountering an ultrasonic wave, the timing of the emitters may be synchronized to correspond with the generation of the ultrasonic wave. In embodiments, the light may be detected only during the first traversal of the ultrasound pulse across the tissue after its transmission. Accordingly, the operation of the analog-to-digital converter 36 may be gated by the ultrasound drive 39 by means of a gate signal. In embodiments, the ultrasound transducer 12 is designed to produce not a beam but a pronounced ultrasound focus at a defined depth and position. By means of a gate signal, the optical signal may be recorded only for the short period of the ultrasound pulse traversing the focus. The ultrasound field may also be chirped. Chirping sweeps the frequency of the ultrasound field so that axial position information is encoded into the Doppler shifted frequency. The repetition of the chirped signal may be controlled by the TPU 28.

In an embodiment, the emitters are manufactured to operate at one or more certain wavelengths. Variances in the wavelengths actually emitted may occur which may result in inaccurate readings. To help avoid inaccurate readings, the sensor assembly 10 may include components such as an encoder 116 that may be used to calibrate the monitor 20 to the actual wavelengths being used. The encoder may be a resistor, for example, whose value corresponds to coefficients stored in the monitor 20. The coefficients may then be used in the algorithms. Alternatively, the encoder 116 may also be a memory device, such as an EPROM, that stores information, such as the coefficients themselves. Once the coefficients are determined by the monitor 20, they may be inserted into algorithms for determining hemodynamic parameters. In an embodiment in which the sensor assembly 10 includes a multiple-wavelength sensor, a set of coefficients chosen for any set of wavelength spectra may be determined by a value indicated by the encoder corresponding to a particular light source in a particular sensor assembly 10. In one embodiment, multiple resistor values may be assigned to select different sets of coefficients. In another embodiment, the same resistors are used to select from among the coefficients for different sources. In embodiments, an encoder 116 may also be associated with an ultrasound transducer 12. For example, the encoder 116 may provide information to a monitor 20 related to the frequency/frequencies of the ultrasound wave generated at the transducer 12 or the incident angle of the wave or the location of the ultrasound transducer 12 relative to the optical emitters 16 or detector 18.

Control inputs 30 may allow a user to interface with the monitor 20. Control inputs 30 may be, for instance, a switch on the monitor 20, a keyboard or keypad, or a port providing instructions from a remote host computer. The monitor 20 may receive user inputs related to the configuration and location of such sensors on the patient. For example, in embodiments, the sensor assembly 10 may be configured to operate on mucosal tissue locations. In other embodiments, the sensor assembly 10 may be configured to operate on a digit. Additionally, patient data may be entered, such as sex, weight, age and medical history data, including, for example, clinical conditions such as COPD that may have an influence on certain hemodynamic parameters.

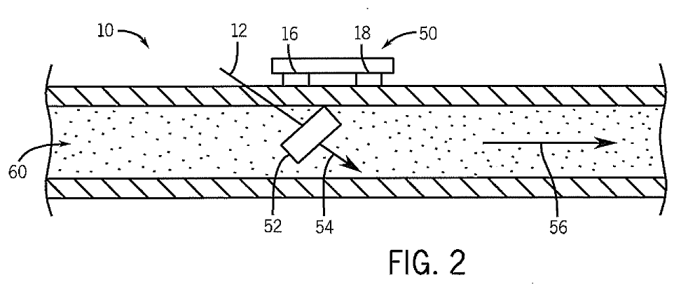

An exemplary sensor assembly 10 is shown in FIG. 2 and includes an optical sensor 50 that includes an emitter 16 and a detector 18. The sensor assembly 10 may also include an ultrasound transducer 12. The optical sensor 50 and the ultrasound transducer 12 may be coupled together in a single sensor unit, such as a disposed on a single sensor body, or may include separate transducer elements and optical elements that may be applied separately to a patient's tissue. In addition, the ultrasound transducer 12 and the optical sensor 50 may be coupled to the monitor 20, either by direct electrical connections or remotely. As shown, the sensor assembly 10 may be applied to a patient's tissue so that light from the emitter may penetrate into a vessel 60. The ultrasound focus area 52 may be selected so that the emitted light may encounter the ultrasound beam 54 and undergo a Doppler shift of a detectable frequency.

In embodiments, the spacing between the emitter 16 and detector 18 may be determined based upon the region of skin or compartment of the body that is to be tested. Generally, for probing of relatively shallow vessels, such as those in certain mucosal tissue, the emitter 16 and detector 18 may be relatively close to one another, while for deeper probing the emitter 16 and detector 18 will be further separated. In certain embodiments, the emitter-detector spacing is between about 1 mm and about 5 mm. In other embodiments, the emitter-detector spacing is between about 2 mm and about 2.5 mm. The spacing of the ultrasound transducer 12 from the optical components of the sensor may be at any distance that allows focusing the ultrasound waves at a proper depth so that the photons may undergo a Doppler shift. In an embodiment, the beam is focused about 0.4 mm into a vessel after the vessel depth has been determined. In one example, the separation of the transducer 12 from the optical components of the sensor is about 2 mm along the flow path of the vessel. The ultrasound focal angle may be about 45 degrees. In embodiments, the ultrasound focal angle is dependent on both the emitter-detector spacing (which determines optical penetration depth) and ultrasound-optical spacing (which is dependent on the location of the vessel and the direction of blood flow, indicated by arrow 56).

The ultrasound transducer 12 may be of any suitable type for converting high-frequency electrical signals into ultrasound waves a beam, which may be transmitted into a patient's tissue. The transducer 12 may also receive the reflected and/or scattered ultrasound waves and convert these into received electrical signals. In an exemplary embodiment, the ultrasound waves are generated using a Doppler or pulsed-wave ultrasound system that includes one or more ultrasonic transducers (such as one or more piezoelectric transducers) for transmitting and/or receiving the one or more ultrasound waves. In embodiments, the one or more ultrasound waves may include a range of carrier frequencies. The frequency may be selected in accordance with one or more transmission characteristics of the blood vessel and/or surrounding tissue/structures. In an exemplary embodiment, the signal frequency may be between about 10 and 40 MHz, inclusively.

The emitter 16 may be configured to transmit electromagnetic radiation, such as light, into the tissue of a patient. The electromagnetic radiation is scattered and absorbed by the various constituents of the patient's tissues, such as red blood cells. A photoelectric detector 18 in the sensor 50 is configured to detect the scattered and reflected light and to generate a corresponding electrical signal. The sensor 50 directs the detected signal from the detector 18 to the monitor 20.

The emitter 16 and a detector 18 may be of any suitable type. For example, the emitter 16 may be one or more laser diodes adapted to transmit one or more wavelengths of light in the red to infrared range, and the detector 18 may one or more photodetectors selected to receive light in the range or ranges emitted from the emitter 16. Alternatively, an emitter 16 may also be a laser diode or a vertical cavity surface emitting laser (VCSEL). An emitter 16 and detector 18 may also include optical fiber sensing elements. An emitter 16 may include a broadband or “white light” source, in which case the detector could include any of a variety of elements for selecting specific wavelengths, such as reflective or refractive elements or interferometers. These kinds of emitters and/or detectors would typically be coupled to the rigid or rigidified sensor via fiber optics. Alternatively, a sensor 50 may sense light detected from the tissue at a different wavelength from the light emitted into the tissue. Such sensors may be adapted to sense fluorescence, phosphorescence, Raman scattering, Rayleigh scattering and multi-photon events or photoacoustic effects. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra. In embodiments, the emitter 16 emits light at a wavelength in the range of about 400 nm to about 800 nm.

The emitter 16 and the detector 18 may be disposed on a sensor housing, which may be made of any suitable material such as plastic, foam, woven material, or paper. Alternatively, the emitter 16 and the detector 18 may be remotely located and optically coupled to the sensor assembly 10 using optical fibers.

The sensor 50 may include a “transmission type” sensor. Transmission type sensors include an emitter 16 and detector 18 that are typically placed on opposing sides of the sensor site. If the sensor site is a fingertip, for example, the sensor assembly 10 is positioned over the patient's fingertip such that the emitter 16 and detector 18 lie on either side of the patient's nail bed. In other words, the sensor 50 is positioned so that the emitter 16 is located on the patient's fingernail and the detector 18 is located 180° opposite the emitter 16 on the patient's finger pad. During operation, the emitter 16 shines one or more wavelengths of light through the patient's fingertip and the light received by the detector 18 is processed to determine various physiological characteristics of the patient. In each of the embodiments discussed herein, it should be understood that the locations of the emitter 16 and the detector 18 may be exchanged. For example, the detector 18 may be located at the top of the finger and the emitter 16 may be located underneath the finger. In either arrangement, the optical sensor 50 will perform in substantially the same manner.

Reflectance type sensors also operate by emitting light into the tissue and detecting the light that is transmitted and scattered by the tissue. However, reflectance type sensors include an emitter 16 and detector 18 that are typically placed on the same side of the sensor site. For example, a reflectance type sensor may be placed on a patient's fingertip or forehead such that the emitter 16 and detector 18 lie side-by-side. Reflectance type sensors detect light photons that are scattered back to the detector 18. A sensor assembly 10 may also include a “transflectance” sensor, such as a sensor that may subtend a portion of a baby's heel.

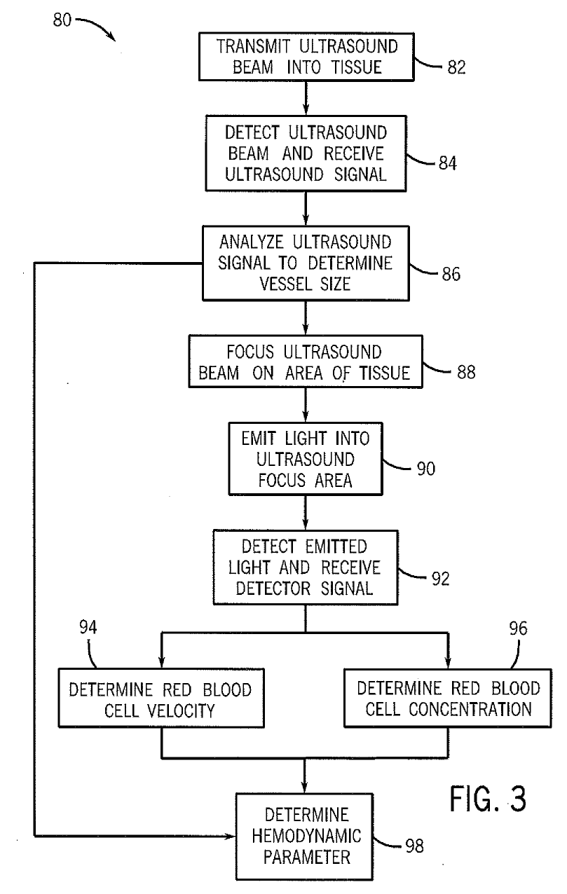

FIG. 3 is a flow chart of an embodiment of a processing method 80 for determining hemodynamic parameters. In the embodiment, an ultrasound beam may be transmitted (block 82) into the tissue of a patient into an area perfused with blood vessels and may be received by a suitable device, such as a monitor 20 (block 84). Next, the ultrasound signal may be processed and analyzed to determine the size of the probed blood vessels (block 86).

In an exemplary embodiment, the ultrasound waves may be generated using a continuous wave, Doppler, pulsed-wave, or pulsed-chirp ultrasound system that includes one or more ultrasonic transducers 12 (such as one or more piezoelectric transducers) for transmitting and/or receiving the one or more ultrasound waves. In one embodiment, the transducer 12 may continuously transmit ultrasound waves and receive the reflected waves. In another embodiment, the transducer 12 may transmit an ultrasound wave of varying frequency over time.

The one or more reflected and/or scattered ultrasound waves are converted into received electrical signals (block 84) in the transducer 12 and may be used to determine one or more characteristics of the vessel (block 86), such as a mean cross-sectional diameter D. In one embodiment, the ultrasound transducer 12 may be capable of generating pulsed waves for a period of time in order to generate electrical signals that include information corresponding to Doppler frequencies. These Doppler frequency shifts of the ultrasound beam are separate from the optical Doppler shift. Each Doppler frequency component in a spectrum of Doppler frequencies provides a measurement of an acoustic power that is proportional to a volume of scatterers in the sample volume that moved through the one or more beams at a corresponding velocity. For backscattering measurements, the Doppler frequency is given by 2(f/c)V cos(θ), where the factor of 2 is associated with round-trip propagation path differences, f is the carrier frequency of an ultrasound wave, c is a speed of sound (ranging from 1470 m/s in water to 4800 m/s in bone), V is the velocity of the scatterers and θ is the incidence angle of the ultrasound beam.

A thickness of the sample volume may be defined using range gating of the one or more reflected and/or scattered ultrasound waves (or the corresponding received electrical signals after transduction) that are received at the transducer 12. A lateral dimension of the sample volume may correspond to widths of the one or more beams. These, in turn, may be an inverse function of an aperture of the one or more transducers 12. Frequency chirping can also be used to define the axial dimension of the volume.

In block 88, the ultrasound transducer 12 focuses the beam into an area that corresponds to a region overlapping the photon distribution generated by the optical emitter 16 in the tissue. The focus of the beam may be modified using a mechanical lens, defocusing, electronic steering, or electronic focusing. At block 90, the optical source emits light into the tissue concurrently with the focused ultrasound beam. The photons of light in the ultrasound focus area 52 undergo a Doppler shift that can be detected at the detector 18, for example using heterodyone techniques. In embodiments, coherent radiation from laser sources may be split into two beams. One beam may be used as a reference oscillator and the other is used to probe the tissue bed. The light returned from the tissue bed is incident on a photodetector with the local oscillator in order to do heterodyne down conversion which yields a beat signal that is proportional to the strength of the absorption at the focus of the ultrasound field. In embodiments, the detector 18 may be a photomultiplier, capable of detecting both Doppler-shifted and non Doppler-shifted light. A frequency selective filter may be used to isolate the Doppler shifted frequencies of interest from the detector, for example with square law detectors. The detector 18 generates a signal at block 92 that may be analyzed at block 94 to provide information about the red blood cell velocity and at block 96 to provide information about the red blood cell concentration.

In block 98 the information from blocks 88, 94, and 96 may be used to calculate a physiological parameter such as hematocrit. The hematocrit (Hct) of vessel 60 can be expressed as NVB(Tc/4)D'L, where VB is the mean volume of a red blood cell. Hence, the hematocrit for any region of vessel 60 can be expressed by the following probability function: F(N)=NVB(Tc/4)D'L where N is a parameter that varies along the vessel length L at any given time, and also varies in time, at any given point along the vessel length L. For example, at any given time, a section of blood vessel 60 may have an average number of red blood cells. The standard deviation of the mean N is proportional to the square root of N, and the coefficient of variation can be calculated as the standard deviation over the mean. Thus, the coefficient of the variation of N may be a function of the Hct and the vessel diameter. In embodiments, the bounding volume may be the ultrasound field itself, if the focus lies within a region within the vessel.

In one embodiment, the photons of light that undergo the Doppler shift may be “tagged.” For example, when photons of light enter a Doppler field of an ultrasound beam that is frequency modulated (i.e., a pulse chirp), the magnitude of the Doppler shift as a function of the frequency modulation may be related to the distribution of photons within the tissue. The optical signal may be detected and processed so as to select a signal component in which the magnitude of the Doppler shift exceeds a predetermined threshold, whereby this threshold may be indicative of photons that have significantly traversed a blood vessel located at or near the ultrasound focus so that the isolated component is very highly indicative of one or more properties of the blood in the vessel. The optical properties of the blood and/or vessel may be more specifically isolated by comparing the selected component to an optical intensity reference including a similarly selected component of an ultrasound-modulated optical signal from a second optical path having similar dimensions (i.e., emitter-detector-transducer spacing and ultrasound focal depth), where the second optical path does not traverse the blood vessel. This optical intensity reference may be derived by moving the same sensor to similar, and preferably adjacent, tissue, or by integrating a second emitter, detector, and/or transducer into the sensor so as to form a second reference path away from the vessel. For instance, a sensor 50 may be constructed so as to define a first emitter-transducer-detector path along the direction of a vessel and a second reference path at a right angle to the vessel.

In one embodiment, the signal at the photodetector 18 includes “speckle.” Speckle is an interference phenomenon that occurs when coherent light (e.g., laser light) is reflected from a rough or multiply scattering sample onto a detection plane. Due to scattering of photons from and within the sample, different photons travel different distances to the detection plane. As a result, the light reflected or backscattered from the sample, if spatially and temporally coherent, interferes at the detection plane, producing a grainy pattern known as “speckle.” In operation, coherent light, such as laser light, from an emitter 16 is transmitted via a beam-splitter through a fixed optical fiber into a patient's tissue. Light remitted from the patient reflects from a mirror 16 into optical fibers to a detector 18. Due to interference, a speckle pattern forms at the detector 18. In embodiments, the detector 18 may include a charge coupled detector array. The resulting speckle pattern is then digitized by an analog-digital converter, and analyzed, such as using the procedures provided in U.S. Pat. No. 7,231,243 to Tearney et at, the specification of which is incorporated by reference for all purposes. herein The speckle pattern may be analyzed to determine certain features of the tissue or vessel. In one embodiment, the speckle pattern may be analyzed to determine blood vessel diameter.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of hemodynamic parameters such as hematocrit, but these techniques may also be utilized for the measurement and/or analysis of other physiological parameters such as pulse oximetly, hemoglobin concentration, or red blood cell count. Rather, the various embodiments may to cover all modifications, equivalents, and alternatives falling within the spirit and scope of the disclosure as defined by the following appended claims

What is claimed is:

1. A monitoring system comprising:
a storage device storing routines for:
receiving a signal from a photodetector;
receiving a signal from an ultrasound transducer;
determining at least one of a blood flow velocity or a red blood cell level based at least in part on the photodetector signal;
determining a blood vessel size based at least on part on the ultrasound transducer signal;
determining a physiological parameter based at least in part on the blood vessel size and one or more of the flow velocity or the red blood cell level; and
a processor capable of executing the stored routines.

2. The system as set forth in claim 1, wherein the physiological parameter comprises a hemoglobin value, a hematocrit value, or a blood pressure value.

3. The system as set forth in claim 1, comprising an emitter capable of emitting light into a tissue and generating the signal at the photodetector.

4. The system as set forth in claim 3, wherein the emitter and the photodetector are spaced about 2 mm to about 3 mm apart from one another.

5. The system as set forth in claim 3, comprising the ultrasound transducer.

6. The system as set forth in claim 5, wherein the ultrasound transducer is capable of being focused at a depth of less than 5 mm from a surface of the tissue.

7. The system as set forth in claim 5, wherein the ultrasound transducer is capable of transmitting a frequency-modulated ultrasound wave.

8. The system as set forth in claim 5, wherein the ultrasound transducer is spaced apart less than 2 mm from either the emitter or the photodetector.

9. The system as set forth in claim 5, wherein the ultrasound transducer is capable of transmitting ultrasound waves into the tissue at the same time the emitter transmits light into the tissue.

10. A method, comprising:
receiving a signal from a photodetector;
receiving a signal from an ultrasound transducer;
determining at least one of a blood flow velocity or a red blood cell level based at least in part on the photodetector signal;
determining a blood vessel size based at least on part on the ultrasound transducer signal;
determining a physiological parameter based at least in part on the blood vessel size and one or more of the flow velocity or the red blood cell level.

11. The method as set forth in claim 10, wherein determining the physiological parameter comprises determining a hematocrit value.

12. The method as set forth in claim 10, wherein determining the physiological parameter comprises determining a blood pressure value.

13. A method comprising:
emitting photons into a blood vessel of a patient's tissue;
focusing an ultrasonic beam into the blood vessel so that a portion of the photons in the blood vessel experience a Doppler shift;
generating a signal related to detected photons at a detector; and
processing the signal to isolate a signal component representative of photons that have undergone a Doppler shift of a magnitude greater than a predetermined threshold; and
analyzing the isolated signal component to determine one or more properties of the blood vessel.

14. The method as set forth in claim 13, comparing the isolated component to a reference signal comprising a different isolated signal component representative of photons that do not traverse the blood vessel.

15. The method as set forth in claim 14, comprising emitting photons into the tissue away from the direction of flow of the blood vessel to generate the reference signal.

16. The method as set forth in claim 13, wherein focusing an ultrasonic beam comprises focusing a frequency-modulated ultrasonic beam.

17. The method as set forth in claim 13, comprising analyzing a speckle pattern of the photons detected at the detector to determine information about the blood vessel.

18. The method as set forth in claim 15, comprising determining a vessel diameter based at least in part on the speckle pattern.

19. The method as set forth in claim 13, comprising determining a concentration of red blood cells based at least in part on the detected photons.

20. The method as set forth in claim 13, comprising determining a concentration of velocity of red blood cells based at least in part on the detected photons.
